(19) United States
(12) Patent Application Publication
(10) Pub. No.: US 2021/0040509 A1
(43) Pub. Date: Feb. 11, 2021

(54) DEHYDRATION ENERGY RECYCLING SYSTEM AND METHOD

(71) Applicant: Bioleap, Inc., Tampa, FL (US)

(72) Inventors: James Knight, JR., Tampa, FL (US); Olan Wayne Mitchell, Sarasota, FL (US)

(73) Assignee: Bioleap, Inc., Tampa, FL (US)

(21) Appl. No.: 16/984,242

(22) Filed: Aug. 4, 2020

Related U.S. Application Data
(60) Provisional application No. 62/882,758, filed on Aug. 5, 2019.

(51) Int. Cl. C12P 7/06; B01D 3/00; B01D 1/28; B01D 5/00

(52) U.S. Cl. CPC C12P 7/06; B01D 3/007; B01D 5/0075; B01D 1/28; B01D 3/001

(57) ABSTRACT

A dehydration system energy recycling system (17) and method whereby latent heat energy is transferred from a high proof vapor produced by a dehydration element (16′) into a lower proof feed mixture received into the dehydration element. The high proof vapor is first compressed (48) downstream of a dehydration apparatus (18) to increase its saturation temperature, and is then condensed to release latent heat energy. The latent heat energy is used to heat the lower proof feed mixture upstream of the dehydration apparatus. A grain-to-alcohol plant incorporating the dehydration system energy recycling system requires little or no virgin boiler steam to drive the dehydration system, while an associated evaporation element (24) of the plant can be driven by heat energy captured in a dryer exhaust energy recycling (DEER) system (40).

This application claims benefit of the 5 Aug. 2019 filing date of U.S. provisional patent application No. 62/882,758.

### FIELD OF THE INVENTION

This invention relates generally to the field of energy management in chemical processing plants, and more specifically in one embodiment to energy management in a corn-to-ethanol plant.

### BACKGROUND OF THE INVENTION

Chemical production processes are designed to optimize thermal efficiency in order to minimize the use and cost of energy. Many chemical processes utilize steam as a working fluid, and many chemical production plants include a boiler system fired by natural gas or other combustible fuel. Different processes within a plant may require steam at different operating conditions, and the plant boiler is designed to provide virgin steam at temperature/pressure conditions adequate to satisfy the most rigorous demand conditions within the plant. Process energy demands requiring less energetic steam conditions are typically satisfied by reusing partially spent energy received downstream of a more rigorous use, or if reuse is not a practical option, by reducing the pressure of virgin steam through a reduction device such as a valve or orifice. When the pressure of virgin steam is reduced, the saturation temperature of the steam is reduced as well.

Steam (water, H₂O) is a well-studied compound and is probably the most common compound used as a heating agent in chemical plants. This is due to the many attributes that steam (water) has including: (a) water/steam is non-toxic and safe; (b) water/steam is cheap and plentiful; (c) water/steam is relatively non-corrosive; (d) water/steam has a relatively high heat capacity and high heat of vaporization/condensation; (e) water is easily transported with known pump technologies; (f) steam travels through piping systems with ease; and many other admirable attributes. One of the most useful attributes of steam as a heating agent in chemical process is the relatively large quantity of heat energy delivered when the steam condenses into a liquid. This energy is known as the heat of vaporization. The temperature at which this heat energy is delivered depends on the pressure at which the steam operates at/in the point of heat exchange. As is true with all gases (including steam) that follow the ideal gas law, gases at higher pressures have higher saturation temperatures than the same gas at lower pressures. The energy known as the heat of vaporization is released when a gas condenses. Condensation almost always occurs at the gas's saturation temperature.

FIG. 1 illustrates a prior art arrangement for energy management in a grain-to-alcohol plant having multiple energy demands. Such plants are known to produce an alcohol product such as ethanol, from an agricultural product such as corn. Dry mill corn-to-ethanol plant 100 includes a fermentation element 10 receiving corn which has been ground, mixed with water and enzymes and sometimes other chemicals, and heated in a process that is well known in the art. The fermentation process produces carbon dioxide and a beer product containing solids, ethanol and water. The beer product is passed to a distillation element 12 where a distillation process removes solids and begins the process of removing the water from the ethanol, producing a low proof ethanol/water mixture (overhead vapors) at typically about 190 proof, and also producing a whole stillage product typically containing about 15% solids. The 190 proof ethanol mixture is produced as a vapor which may be processed through a condenser 14 to form a 190 proof liquid.

Additional water is removed from the 190 proof mixture in a dehydration element 16 which includes a dehydration apparatus 18, such as one or more molecular sieves (e.g. as sold by Vogelbusch USA, Inc.) or selective membrane separation units (e.g. as sold by Whitefox Technologies Canada Ltd.) or other device that preferentially removes water from the mixture. A dehydration element of a grain-alcohol plant may also include components that prepare the alcohol (ethanol) mixture received from the distillation element for the dehydration process, such a heaters, vaporizers, etc. Because the dehydration apparatus 18 typically operates on a high pressure superheated vapor, the 190 proof mixture is heated/vaporized as necessary in a heater 20 which usually receives high pressure virgin steam (having a correspondingly high saturation temperature) from a boiler element 22 (which is separate from the dehydration element 16). The dehydration apparatus 18 produces an ethanol product vapor at close to 200 proof. Other known technologies delivering/depositing energy into the 190-proof mixture in the dehydration element 16 all source their energy from outside the dehydration element 16. The dehydration element 16 of FIG. 1 excludes the distillation element 12 and the evaporation element 24 discussed below.

The whole stillage produced by the distillation element 12 is processed through an evaporation element 24 which may include a centrifuge 26 producing a wet cake typically containing about 35% solids and a thin stillage containing about 5% solids. A multi-effect evaporator 28 is used to further process the thin stillage to produce a syrup and water (vapor). The water vapor produced by the evaporator can be recycled back to the front end of the plant (not illustrated). The wet cake and syrup are dried in a dryer element 30, typically in a gas-fired drum dryer 32, to produce a dried distiller's grain (DDG) product which can be sold as animal feed.

While the heat energy required for the dehydration step is usually provided through a heater receiving high pressure virgin steam (having a high saturation temperature), the energy for the various stages of the evaporator is provided at a plurality of different temperature/pressure conditions, thereby allowing the heating agent to have a relatively lower saturation temperature. To drive a multi-effect evaporator 28, it is known to utilize the 200 proof ethanol vapor output from the dehydration apparatus 18 (having a relatively lower saturation temperature than the 200 proof ethanol vapor within the dehydration apparatus), either directly or indirectly, along with intermediate pressure/temperature steam (having a relatively intermediate saturation temperature) provided from the boiler element through a pressure reduction device 34. As it passes through the evaporator, the 200 proof ethanol product vapor is cooled and condensed, thereby releasing its heat of vaporization (heat of condensation), in order to form 200 proof ethanol product liquid, which may be collected in a tank or other liquid collection device 36. The liquid may then be further cooled in a non-contact (typically liquid/liquid) regenerative economizer (heat exchanger) 38 providing heat exchange with the relatively cooler 190 proof ethanol feed mixture received from the distillation element.

FIG. 2 illustrates a recent improvement in energy management technology for grain-to-alcohol plants. Corn-to-ethanol plant 200 of FIG. 2 includes most of the elements of plant 100 of FIG. 1, with like elements being numbered consistently in both figures. But unlike plant 100 of FIG. 1 which provides heat energy to the evaporation element from the dehydration element and from the boiler through a pressure reduction device, plant 200 of FIG. 2 provides heat energy to the evaporation element from the dehydration element and from a dryer exhaust energy recovery (DEER) system 40. The DEER system extracts heat energy from the exhaust gas exiting the dryer by passing the hot exhaust gas through the hot side of a non-contact economizer 42 at any location upstream of the release of the exhaust to the surrounding environment. Hot water produced on the cold side of the non-contact economizer 42 is converted to low pressure steam in a flash vessel 44. The low pressure steam is then mixed with high pressure steam from the boiler element 22 in a thermocompressor 46 to form intermediate pressure steam for delivery to the evaporator 28. By capturing heat energy from the dryer exhaust gas which otherwise would have been lost to the atmosphere, the DEER system 40 reduces the amount of virgin steam required to meet the evaporator demand. For every one BTU of thermal energy demand satisfied by a DEER system, about one-half BTU is captured from the exhaust gas, and thus about one-half BTU of virgin steam is saved, thereby improving the plant's overall efficiency and reducing the plant's operating cost and carbon footprint. Non-limiting examples of such a dryer exhaust energy recovery system may be better understood by reference to U.S. Pat. Nos. 9,989,310 B2 and 10,345,043 B2, both assigned to Bioleap, Inc. and incorporated by reference herein.

### BRIEF DESCRIPTION OF THE DRAWINGS

The invention is explained in this description in view of the drawings that show:

FIG. 1 is a schematic illustration of a prior art corn-to-ethanol plant.

FIG. 2 is a schematic illustration of an improved prior art corn-to-ethanol plant.

FIG. 3 is a schematic illustration of an embodiment of the present invention in a corn-to-ethanol plant.

FIG. 4 is a schematic illustration of another embodiment of the present invention in a corn-to-ethanol plant.

### DETAILED DESCRIPTION OF THE INVENTION

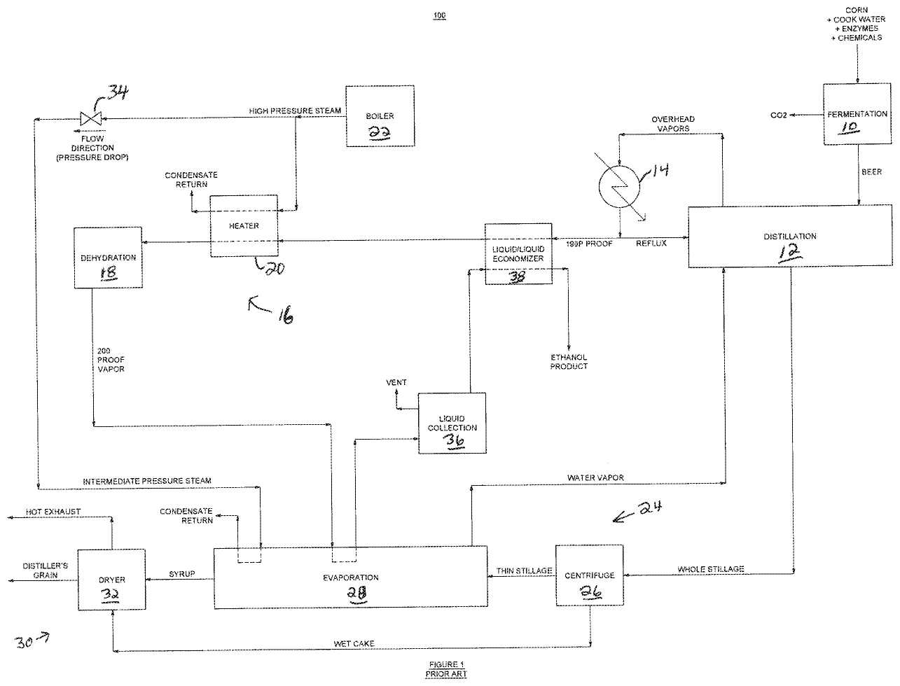

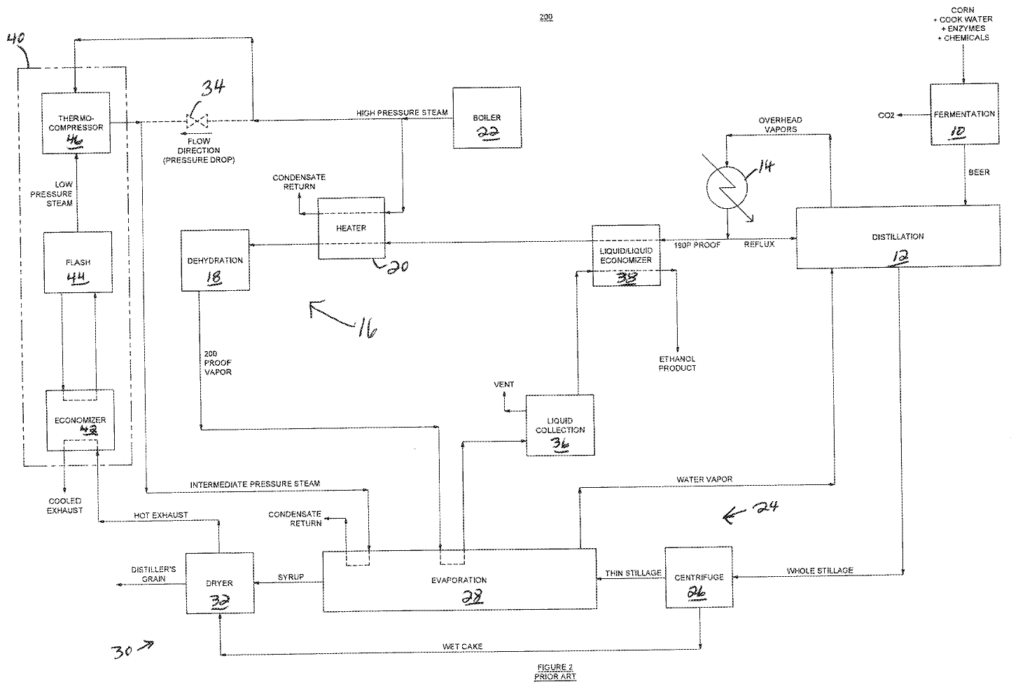

As part of their ongoing efforts to improve energy management in chemical processing plants, the present inventors have discovered that instead of transferring energy contained in the dehydrated ethanol product produced in a corn-to-ethanol plant to an evaporation system/element of the plant, as is done in the prior art, further energy efficiency gains and plant design options are made possible by recycling that heat energy within the dehydration system itself. Prior art plant designers have recognized that the temperature/pressure conditions of the 200 proof vapor exiting the dehydration apparatus 18 of FIGS. 1 and 2 (approximately 290° F. and 50 psig, for example) do not support the reintroduction of that energy back into the dehydration element, but those conditions are ideal as a heat input source to the evaporation system. Thus, prior art corn-to-ethanol plants direct the dehydrated ethanol vapor product through the evaporation element 24 (as illustrated in FIGS. 1 and 2) to provide heat energy input to the evaporators 28 and to cool/condense the high proof ethanol product in preparation for commercial delivery. Other prior art technologies have identified alternative uses for this energy, including as an energy source to drive a reboiler in distillation (not illustrated).

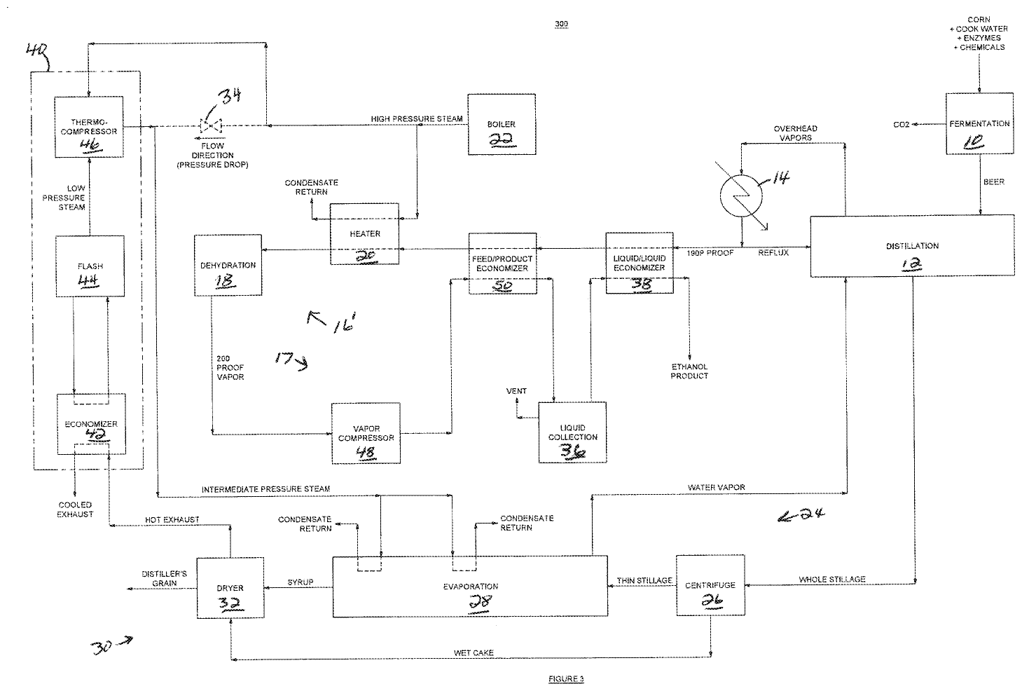

FIG. 3 illustrates a grain-to-alcohol plant embodiment of the present invention wherein heat energy output from the dehydration apparatus 18 is recycled within the dehydration element 16′ rather than being repurposed to the evaporator 28. The corn-to-ethanol plant 300 of FIG. 3 includes most of the elements of the plants of FIGS. 1 and 2, with like elements being numbered consistently in all of the figures. In addition, the dehydration element 16′ of plant 300 includes a novel dehydration energy recycling system 17 which greatly reduces or eliminates the amount of virgin steam that must be supplied to drive the dehydration system operation.

The relatively lower proof (e.g. 190 proof) ethanol feed mixture supplied from the distillation element is relatively cool (typically 150° F.) even after passing through the known regenerative heat exchanger 38. That mixture must be heated, and in some plants heated and vaporized, to facilitate proper operation of the dehydration apparatus 18. In the prior art, this heat energy is provided entirely from virgin boiler steam via a heat exchanger 20 or from other sources in the plant outside of the dehydration element 16. Rather than utilizing virgin steam and/or another energy source outside the dehydration system as the sole source(s) of this energy, plant 300 captures thermal energy from the high proof vapor product produced by the dehydration apparatus 18, in spite of the problematic fact that the temperature/pressure conditions of the high proof vapor product produced by the dehydration apparatus 18 (for example 290° F. and 50 psig) are inadequate for that purpose. To solve that problem, plant 300 innovatively incorporates a mechanical vapor compressor 48 or other similarly functioning device downstream of the dehydration apparatus 18 to increase the pressure (and temperature) of the high proof vapor product, such as to 350-400° F. and 150 psig. Thermal vapor recompression may be used in lieu of or in combination with the mechanical vapor recompression. The recompressed vapor is then directed to a hot side (product side) of a feed/product economizer 50 for non-contact heat exchange therein with a flow of the incoming (feed side) lower proof mixture received from the distillation element 12, wherein the high proof vapor is at least partially (preferably mostly or fully) condensed and the incoming feed product mixture is heated and at least partly or entirely vaporized. The recompressed vapor is provided to the hot side of the feed/product economizer 50 at a saturation temperature that is higher than the operating temperature of the low proof mixture on the cold side of the economizer 50 in order to provide effective heat transfer and condensation of the high proof vapor. The recompressed vapor may have a saturation temperature that is at least about 10° F. above the saturation temperature of the low proof ethanol vapor taken at the inlet to the dehydration apparatus (the physical location where the water is separated from the ethanol, such as in a molecular sieve bed).

The prior art designs of FIGS. 1 and 2 have recognized the benefit of extracting energy from the high proof dehydrated ethanol product, and they have applied that energy to a lower level (lower saturation temperature requirement) energy demand of the plant, i.e. the evaporation system. Mechanical vapor recompression is an energy-additive process, and in spite of the fact that it is counterintuitive to add energy to a fluid from which you want to remove energy, the present inventors have innovatively utilized a vapor recompression step to facilitate the recycling of energy within the dehydration element 16′ itself. By raising the pressure of the high proof vapor product to a level such that the saturation temperature of the high proof vapor is above the operating temperature of the incoming low proof feed mixture, latent heat energy of the high proof vapor is made useful within the dehydration system itself. Not only is the relatively small amount of energy added by the mechanical vapor recompression step available for recycling through the feed/product economizer 50, but the recompression step achieves conditions in the 200 proof product vapor which allow it to be condensed by heat exchange with the incoming 190 proof feed product, thereby releasing the latent heat energy of the 200 proof vapor. As a result, the only virgin energy required to support an ongoing dehydration process is the replenishment of energy lost to the ambient environment or to venting, or to other unit operations, etc. The steam heater 20 of prior art designs may be used to provide this makeup energy, with the steam heater 20 and the feed/product economizer 50 being connected in series (in either order) or in parallel, depending upon the particulars of a specific plant design. Alternatively or in combination, an electrical heater, heat pump, steam jacket, and/or other known heat exchange technology/method (not illustrated) may be used to add makeup heat to the system in conjunction with the dehydration energy recycling system 17.

To optimize the benefit obtained from the dehydration energy recycling system 17, the high proof vapor product should preferably remain in the vapor state until condensed in the feed/product economizer 50. Other operations affecting the high proof vapor that do not cause a phase change are not deleterious to the energy efficiency gain of this invention, such as heating or cooling or combining the vapor with another fluid or storing the vapor or changing the pressure without a resulting phase change.

While the prior art regenerative heat exchanger 38 may continue to be used in some embodiments, that device is designed to transfer only sensible heat, since the hot side fluid is received after having been condensed, either in the evaporator system for the prior art designs of FIGS. 1 and 2, or in the feed/product economizer of FIG. 3. The dehydration energy recycling system 17 enables the recycling of both latent and sensible heat from the dehydrated high proof vapor product, with the energy content of the latent heat being on the order of 50-500 times greater than the specific heat energy available in that fluid for productive use in the plant. Once the high proof product has been condensed in the feed/product economizer 50, it may be directed to a liquid collection tank 36 for further processing, such as to be denatured prior to commercial sale. Once the high proof product has been condensed, sensible heat from the liquid may be recovered in a further heat exchange operation at any desired point in the downstream processing of that fluid. The inventors understand that an advantageous heat exchange operation for the reuse of specific heat energy from the high proof liquid may occur in a regenerative economizer 38 wherein the high proof liquid exchanges heat in an indirect liquid/liquid heat exchange operation with the liquid low proof ethanol mixture, the liquid low proof ethanol mixture receiving this heat energy prior to the low proof ethanol entering the feed/product economizer 50.

In plant 300 there is no energy provided to the evaporation element 24 from the dehydration element 16′. That energy demand is satisfied by the dryer exhaust energy recycling (DEER) system 40 (and may be supplemented with virgin boiler steam in other embodiments). The dehydration energy recycling system 17 does not compete with the DEER system 40, nor do those two systems deposit energy to a common production unit operation. The dehydration energy recycling system 17 allows virgin steam to be used only for demands which are not practically satisfied by the DEER system 40. In prior art plants which incorporate a DEER system, such as plant 200 of FIG. 2, there is usually capacity in the DEER system 40 which exceeds the demand of the evaporation element 24, and that excess capacity often goes unused. In prior art plants 100 and 200, much of the demand for energy in the evaporation element 24 is satisfied by the condensation of the 200 proof vapor in a first effect evaporator 28. Because the energy recycled in the dehydration element 16′ of FIG. 3 is not being delivered to the evaporation element 24, the DEER system 40 can be used to satisfy that demand, allowing the highly efficient DEER system 40 to work more closely to its full capacity. One skilled in the art will recognize that the level of efficiency gain may vary somewhat from plant to plant, but generally for every one BTU of thermal energy demand provided by a DEER system 40, only about one-half BTU of virgin steam is required from the boiler system 22, with the other one-half BTU being recovered from the dryer exhaust gas. Thus it is advantageous to maximize the utilization of a DEER system. For each BTU of energy recycled by the dehydration energy recycling system 17 of plant 300, one BTU less virgin steam energy is required for the dehydration system 16′ and one additional BTU of energy must be provided by the DEER system 40 to the evaporation element 24, but only one-half BTU of virgin steam is required to produce that extra one BTU in the DEER system 40. Thus, there is a net saving for the plant of one-half BTU for every BTU recycled by the dehydration energy recycling system 17. Even in prior art plants where a DEER system 40 is already being operated at or near capacity, there is usually additional unextracted heat energy available in the dryer exhaust gas at some location in the exhaust gas flow between the dryer 32 and the downstream atmospheric stack (not illustrated). For those plants, the existing DEER system 40 may be upgraded and/or it may be expanded; i.e. it may be extended to extract more heat energy from the same point or to extract energy from a different point in the exhaust gas flow path, such as by enlarging or adding an additional DEER economizer 42. The economizer of a DEER system in accordance with embodiments of the present invention may be positioned at any appropriate location within the dryer exhaust gas flow path. Thus, an additional economizer used to augment dryer exhaust energy recovery in accordance with embodiments of the present invention may be located at the location of an original economizer 42 or at a new location, such as downstream of any source of heat that is added to the dryer exhaust gas flow downstream of the dryer 32. For example, if an existing plant positions its DEER economizer 42 upstream of a thermal oxidizer (not illustrated) because that location optimizes the economics of the system, a second DEER economizer (not illustrated) may be added at that location to work in parallel or in series with the original economizer 42, and/or a supplemental DEER system economizer can be added downstream of the thermal oxidizer, to increase the overall capacity of the DEER system 40 in order to enable the dehydration energy recycling system 17 to achieve its maximum efficiency gain.

Plant 300 requires only known types of plant process instrumentation and control systems. Overpressure protection in the dehydration energy recycling system 17 may be accomplished by known methods, such as by actively controlling a variable frequency drive motor associated with the compressor 48, by venting excess pressure to the atmosphere (with resulting product loss), or by venting excess pressure to a non-contact heat exchanger within the plant (without product loss). Such non-contact heat exchanger may utilize any cooling source available in the plant, including the plant's cooling utility service, cooling tower water or well water, cook water, and/or thin or mid stillage. Embodiments of the invention may utilize an evaporator or a primary condenser in the distillation system (not illustrated) for venting and pressure control of the dehydration energy recycling system.

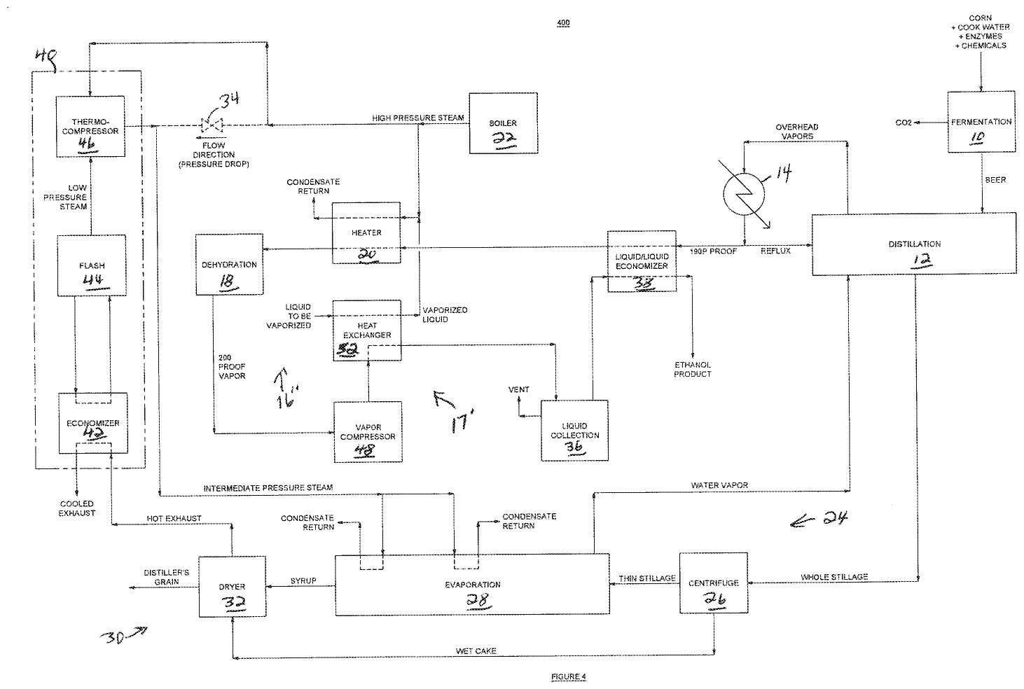

In another embodiment of a dehydration energy recycling system 17′, the corn-to-ethanol plant 400 of FIG. 4 includes most of the elements of the plants of the other figures, with like elements being numbered consistently in all of the figures. Rather than directing the recompressed high proof vapor of the dehydration element 16″ to a feed/product economizer 50, as in FIG. 3, the recompressed vapor in plant 400 is directed to a hot side of a non-contact heat exchanger 52 to be condensed and to produce steam on a cold side of the non-contact heat exchanger 52. This embodiment may be particularly advantageous when back fitting the present invention into an existing plant. The steam produced in the non-contact heat exchanger 52 is then directed to an existing or additional steam heater 20, either via a separate inlet (not illustrated) or by being connected to the steam feed line upstream of the heater, with valving (not illustrated) as appropriate.

One skilled in the art will appreciate that the process conditions and performance levels described above are by way of example only and may vary from plant to plant. Further, the processes of condensation and vaporization are described as they occur on a macro scale within an operating plant, recognizing that on a micro scale there may be nucleate or bulk vaporization/condensation occurring under very local conditions within a particular component that does not significantly impact the overall plant operation (for example, the phenomenon of absorption and adsorption; or for another example, the formation of condensation on the inside walls of pipes as energy is lost to the environment and subsequent re-vaporization as pressure conditions change inside the pipe, purposefully or accidentally). Moreover, some designs may purposefully or accidently result in somewhat less than 100% of the vapor being condensed in the feed/product economizer 50 or heat exchanger 52, thereby recycling some but not all of the available latent heat energy, as well as some, little or no specific heat, depending upon the particular plant design. The type of vapor compression/pressure increasing device used is not critical to the invention, nor is the particular design of the feed/product economizer critical, with any passive or active economizer design that provides the desired condensation and heat transfer being acceptable. The term “element” as used herein is meant to include a single component or a system of interconnected components performing or facilitating a particular function, depending upon the plant design.

While in the embodiments of FIGS. 3 and 4 the dryer exhaust energy recovery system 40 is configured to satisfy all of the first effects energy demand of the evaporation element 28 and there is no transfer of energy from the dehydration element to the evaporation element, one skilled in the art can envision embodiments where the 200 proof vapor remains in heat exchange relationship with the evaporation element as illustrated in FIGS. 1 and 2, but only a portion of the latent heat energy of the 200 proof vapor is transferred there between, less than the quantity of latent heat energy that is transferred in the prior art designs, with the remainder being available for recycling within the dehydration element as illustrated in FIGS. 3 and 4. An exemplary prior art corn-to-ethanol plant may generate a first effects energy demand of about 200 MMBTU/hr of thermal energy to be sourced from outside of the evaporator element to drive its first effect evaporator(s). Forty percent of that first effects energy may be provided from the dehydration element of the plant via the 200 proof vapor, and 60% of that energy may be provided by boiler steam (or by boiler steam in combination with steam produced in a dryer exhaust energy recovery system). Recycling at least some of the latent heat energy of the high proof product vapor within the dehydration element itself, as described herein, results in less energy being available to satisfy the first effects energy demand, but advantageously, that energy demand can be satisfied by the highly efficient dryer exhaust energy recovery system. For example, a dryer exhaust energy recovery system may be configured to provide a quantity of heat energy to the evaporation element adequate to satisfy at least 80% of the first effects energy demand; i.e. to reduce the portion of energy provided to the first effect evaporator(s) from the dehydration element to no more than 20% of the total required to drive the first effect evaporator(s). Other embodiments of the invention may reduce the portion of energy provided to the first effect evaporator(s) from the dehydration element to no more than 10% or no more than 5% of the total required to drive the first effect evaporator(s). Such embodiments may be less beneficial than the embodiments of FIGS. 3 and 4, where no energy recovered from the dehydration element is used to satisfy the first effects energy demand, because such embodiments would require fluid system connections between the dehydration element and the evaporation element similar to those illustrated in FIGS. 1 and 2, as well as requiring a dehydration system energy recycling arrangement similar to those of FIG. 3 or 4. However, such embodiments may be advantageous when implementing the present invention into an existing plant.

The terms “system” and “element” are generally used interchangeably herein, although one skilled in the art will recognize that either may include a single or multiple mechanical components in various embodiments, along with related support equipment such as piping, instrumentation and control.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The invention claimed is:

1. A grain-to-alcohol plant comprising a fermentation element, a distillation element, a dehydration element including a dehydration apparatus, an evaporation element generating a first effects energy demand, a boiler element and a dryer element, the plant further comprising:
a dehydration energy recycling system configured to recycle latent heat energy from vapor product produced by the dehydration apparatus back into feed product flowing to the dehydration apparatus, the recycled latent heat energy thereby being unavailable for use in satisfying the first effects energy demand; and
a dryer exhaust energy recovery system configured to provide heat energy to the evaporation element to satisfy at least 80% of the first effects energy demand.

2. The plant of claim 1, further comprising:
the dehydration element configured to provide no energy to the evaporation element; and
the dryer exhaust energy recovery system configured to satisfy all of the first effects energy demand.

3. The plant of claim 1, further comprising:
an economizer comprising a cold side in fluid communication to receive the feed product upstream of the dehydration apparatus and a hot side in fluid communication to receive the vapor product downstream of the dehydration apparatus; and
a vapor compressor in fluid communication between the dehydration apparatus and the hot side of the economizer and operable to increase a pressure of the vapor product flowing to the hot side of the economizer.

4. The plant of claim 1, further comprising:
a vapor compressor in fluid communication downstream of the dehydration apparatus and operable to increase a pressure of the vapor product produced by the dehydration apparatus;
a non-contact heat exchanger comprising a hot side in fluid communication with the vapor compressor for receiving the increased-pressure vapor product and a cold side producing steam; and
a steam heater comprising a hot side in fluid communication with the cold side of the non-contact heat exchanger to receive the steam and a cold side in fluid communication to receive the feed product upstream of the dehydration apparatus.

5. A system for a corn ethanol plant, the system comprising:
a means for recycling at least a portion of latent heat energy available in an ethanol product vapor produced by a dehydration apparatus of the plant back into an ethanol feed product supplied to the dehydration apparatus, whereby the recycled portion of the latent heat energy is thus not available for use in satisfying a corresponding portion of a first effects energy demand of the plant; and
a means for satisfying the corresponding portion of the first effects energy demand with heat energy captured from exhaust produced by a dryer of the plant.

6. The system of claim 5, wherein the means for satisfying the corresponding portion of the first effects energy demand is configured to supply at least 80% of the first effects energy demand.

7. The system of claim 5, wherein the means for satisfying the corresponding portion of the first effects energy demand is configured to supply all of the first effects energy demand.

8. The system of claim 5, wherein the means for recycling further comprises:
an economizer comprising a cold side in fluid communication to receive the ethanol feed product upstream of the dehydration apparatus and a hot side in fluid communication to receive the ethanol vapor product downstream of the dehydration apparatus; and
a vapor compressor in fluid communication between the dehydration apparatus and the hot side of the economizer and operable to increase a pressure of the vapor product flowing to the hot side of the economizer.

9. The system of claim 5, wherein the means for recycling further comprises:
a vapor compressor in fluid communication downstream of the dehydration apparatus and operable to increase a pressure of the ethanol vapor product produced by the dehydration apparatus;
a non-contact heat exchanger comprising a hot side in fluid communication with the vapor compressor for receiving the increased-pressure ethanol vapor product and a cold side producing steam; and
a steam heater comprising a hot side in fluid communication with the cold side of the non-contact heat exchanger to receive the steam and a cold side in fluid communication to receive the ethanol feed product upstream of the dehydration apparatus.

10. A method of recycling energy in a grain-to-alcohol plant, the plant comprising a fermentation element, a distillation element, a dehydration element including a dehydration apparatus, an evaporation element generating a first effects energy demand, a boiler element and a dryer element, the method comprising:
recycling at least a portion of latent heat energy available in a product vapor produced by the dehydration apparatus back into feed product supplied to the dehydration apparatus, whereby the recycled portion of the latent heat energy is made not available for use in satisfying a corresponding portion of the first effects energy demand; and
satisfying the corresponding portion of the first effects energy demand with heat energy captured from exhaust produced by the dryer element.

11. The method of claim 10, further comprising satisfying at least 80% of the first effects energy demand with the heat energy captured from the exhaust produced by the dryer element.

12. The method of claim 10, further comprising satisfying all of the first effects energy demand with the heat energy captured from the exhaust produced by the dryer element.

13. The method of claim 10, further comprising:
directing the feed product through a cold side of an economizer upstream of the dehydration apparatus;
directing the product vapor through a hot side of the economizer downstream of the dehydration apparatus; and
compressing the product vapor upstream of the hot side of the economizer to raise its saturation temperature.

14. The method of claim 10, further comprising:
compressing the vapor product downstream of the dehydration apparatus;
directing the compressed vapor product through a hot side of a non-contact heat exchanger to release the recycled portion of the latent heat energy;
using the released recycled portion of the latent heat energy to form steam in a non-contact heat exchanger; and
using the steam to heat the feed product upstream of the dehydration apparatus.

15. The method of claim 10, further comprising:
compressing the vapor product downstream of the dehydration apparatus to increase its saturation temperature; and
condensing the compressed vapor product at its increased saturation temperature to release the recycled portion of the latent heat energy.
